(19) United States
(12) Patent Application Publication
(10) Pub. No.: US 2026/0033976 A1
(43) Pub. Date: Feb. 5, 2026

(54) POST-SURGICAL RHINOPLASTY COMPRESSION MASK WITH ADJUSTABLE FITTING

(71) Applicant: Emily Hedrick, St. Petersburg, FL (US)

(72) Inventor: Emily Hedrick, St. Petersburg, FL (US)

(21) Appl. No.: 18/789,865

(22) Filed: Jul. 31, 2024

(51) Int. Cl. A61F 5/08

(52) U.S. Cl. CPC A61F 5/08

(57) ABSTRACT

A post-surgical rhinoplasty compression mask designed to improve patient comfort and healing outcomes following nasal surgery is provided. The mask comprises a hard plastic shell and a removable insert that adapts to the patient's facial contours. The mask is secured on the user's head with Velcro straps, which provide a custom fit and ensure the mask remains stable during use. The components of the mask can be individually removed, replaced, and sterilized, allowing for easy maintenance and optimal hygiene. The mask is designed to provide a comfortable, secure, and therapeutic environment conducive to efficient healing post-rhinoplasty, thereby simplifying the recovery process and enhancing patient comfort.

### FIELD OF THE INVENTION

The present invention relates to the field of medical devices and therapeutic accessories. More specifically, the invention pertains to post-operative recovery aids designed to enhance comfort, promote healing, and contribute to optimal surgical outcomes. Even more specifically, the invention is directed at a novel and improved mask for post-rhinoplasty care, designed to alleviate the discomfort, pain, and swelling often experienced by patients after undergoing nasal surgery.

### BACKGROUND OF THE INVENTION

Rhinoplasty, commonly known as nose reshaping surgery, is one of the most frequently performed plastic surgeries worldwide. It is executed for various reasons, ranging from cosmetic modifications to medical interventions for improving breathing functionality. Despite the high frequency of this procedure, managing the post-operative recovery phase can present challenges for both medical practitioners and patients alike.

Typically, the recovery process from a rhinoplasty procedure entails dealing with swelling, bruising, and discomfort around the surgical site. The process requires diligent care to ensure proper healing, avoid infections, and maintain the intended reshaping of the nasal structure.

Traditionally, this post-operative care involves the use of surgical tape and massage to apply pressure to the nose and compress the tip to reduce swelling. However, these traditional methods have several limitations. First, the tape needs to be replaced daily, which requires the old tap to be removed daily. Removing the tape improperly can cause more swelling, negating the purpose of the tape. Massage can be a useful tool for moving out fluid in the nose, however some surgeons do not suggest it as aggressive massage could cause more scar tissue.

Moreover, in the aftermath of a rhinoplasty procedure, it is imperative to maintain the newly formed shape of the nose while it heals. To address this, nasal splints are usually employed. These splints help maintain the structure and prevent unintentional deformations due to movements or pressure changes. While these devices are generally effective, they come with their own set of drawbacks. Nasal splints are often generic and do not account for the individual differences in nasal morphology or the specific reshaping achieved by the rhinoplasty. Furthermore, they may cause discomfort due to ill-fit and lack of adjustability, which can hinder the patient's compliance with the recommended post-operative care regimen.

However, the use of multiple different devices for each aspect of care can lead to a complex and demanding care routine. The patient may need to constantly juggle between cold compresses, nasal splints, and potentially other devices to meet their post-operative care needs. This complexity can not only burden the patient but also increase the likelihood of non-compliance, leading to less than optimal surgical outcomes and potentially longer recovery times.

Therefore, there is a pronounced need in the field for an improved, integrated post-operative care device that addresses these challenges. A device that can deliver a sustained and evenly distributed pressure effect to alleviate swelling, a mechanism to provide structural support to the newly formed nasal shape, and an option for patient-adjusted fitting for enhanced comfort is much needed. Such a device could simplify the post-operative care regimen, improve patient compliance, and ultimately contribute to better surgical outcomes.

### SUMMARY OF THE INVENTION

The present invention aims to address the existing needs in the field of post-rhinoplasty care. It combines multiple therapeutic features into one easy-to-use device, designed to enhance the patient's comfort and convenience during the recovery process. Through innovative design and materials, the invention integrates compression and structural support functionalities, while allowing user-adjustability for a custom fit, and presents a unique and improved solution for post-operative rhinoplasty care.

The present invention is a post-surgical rhinoplasty compression mask designed to provide a comprehensive and integrated solution for post-rhinoplasty care. The mask comprises a hard plastic shell, housing an internal, removable insert made of a sponge-like material, which comfortably contours to the shape of the patient's nose. Within this hard plastic shell, the sponge-like insert is incorporated to consistently alleviate swelling and provide relief. Further aiding the adaptation to individual morphology, the insert features a series of metal wire casings allowing the patient to mold it to their specific nose shape. In some embodiments, atop the insert, an adjustable nose pad applies additional, customizable pressure to the sides and the bridge of the nose. This mask is secured using straps with Velcro that surround the patient's face and the back of their head, providing necessary support and stability, while allowing for adjustability to different head sizes.

The present invention addresses several limitations of prior art, offering significant improvements for post-rhinoplasty care. It combines compression and support functionalities into one single, user-friendly device, reducing the complexity of the post-operative care routine and increasing the likelihood of patient compliance. The adjustable nature of the mask and the moldable insert ensure a custom fit, mitigating the discomfort often associated with generic nasal splints and offering enhanced comfort to the patient. The mask's secure yet adjustable fitting ensures stability, providing structural support to the newly formed nasal shape during the critical healing period. Overall, the present invention simplifies and enhances post-rhinoplasty care, promoting better surgical outcomes and improved patient experiences.

In a first implementation of the invention, a post-surgical rhinoplasty compression mask comprises:
a hard plastic shell forming an exterior structure of the mask;
a removable insert designed to fit within the plastic shell;
a plurality of straps integrated with said plastic shell, the plurality of straps designed to surround a face and back of a head of the user, providing support and stability to the mask and allowing size adjustability to fit different head sizes.

In some aspects, the mask may further comprise a removable nose pad located on a top of the insert, with adjustable side pressure capabilities for applying additional pressure to sides and a bridge of a nose of a user.

In some aspects, the removable insert may be a sponge-like insert comprising a series of metal wire casings, allowing the insert to be shaped to the contour of a nose of the user.

In some aspects, the sponge-like insert may be composed of a breathable material.

In some aspects, the breathable material facilitates adequate airflow, reducing heat buildup and perspiration.

In some aspects, the metal wire casings within the sponge-like insert are made from a rust-resistant metal.

In some aspects, the metal wire casings are evenly distributed throughout the sponge-like insert.

In some aspects, the removable insert may be a cooling gel insert.

In some aspects, the cooling gel insert may comprise a removable cooling gel enclosed in a leak-proof container.

In some aspects, the removable cooling gel may be non-toxic and safe for skin contact.

In some aspects, the plurality of straps may comprise Velcro fasteners.

In some aspects, the hard plastic shell may be lightweight and hypoallergenic.

In some aspects, the removable nose pad is constructed from a soft, skin-friendly material.

In some aspects, the removable nose pad comprises an embedded mechanical system that facilitates adjustable side pressure capabilities of the nose pad.

In some aspects, the mechanical system comprises a series of adjustable tension springs.

In some aspects, the straps are adjustable in length.

In some aspects, the Velcro fasteners are distributed along the full length of the straps.

In some aspects, the compression mask can be used to manage pregnancy swelling. It is very common to have swelling in certain areas of the body, in particular the nose, during the pregnancy, therefore the compression mask disclosed herein may be used to alleviate the nose swelling during the pregnancy.

In some aspects, the compression mask may be used to manage or alleviate swelling of the nose post any nose injury.

In some aspects, the cooling gel insert, and the sponge-like insert may be interchangeable.

In some aspects, the compression mask is available in multiple sizes to accommodate a range of face and head dimensions.

In some aspects, the compression mask is sterilizable for multiple usages.

In some aspects, the compression mask may further comprise a user-adjustable chin strap for additional support and stability.

In some aspects, the compression mask may further comprise an integrated temperature sensor for monitoring and displaying the temperature of the cooling gel.

In another aspect, the compression mask may be designed to allow unrestricted vision, breathing, and oral communication while being worn.

In some aspects, the compression mask may comprise a mesh structure to support the back of head of the user.

In some aspects, the compression mask may comprise a foam structure to support the back of head of the user.

In some aspects, the mesh structure or the foam structure may be an extension of the plurality of the straps comprising Velcro fasteners.

In another aspect, a kit is provided. The kit comprises the post-surgical rhinoplasty compression mask having any of the features described above, a user manual for instructing patients on how to use and adjust the mask, and a carrying case for storage and transportation of the mask.

In another implementation of the invention, a method of using a post-surgical rhinoplasty compression mask comprises:
adjusting a shape of a removable sponge-like insert, which contains a series of metal wire casings, to fit the contour of a nose of a user; and
securing the mask onto a face of the user using straps equipped with Velcro fasteners, wherein the straps surround the face and back of a head of the user, providing support and stability while allowing for adjustability to fit different head sizes.

In an aspect, the method may further comprise replacing the sponge-like insert with a cooling gel insert that comprises cooling gel to provide a cooling effect.

In some aspects, the method may further comprise periodically checking the cooling gel to maintain consistent temperature and replacing the cooling gel as necessary;

In some aspects, the method may further comprise positioning a removable nose pad on top of the sponge-like insert, with the adjustable side pressure applied to sides and a bridge of the nose of the user.

In some aspects, the method may further comprise adjusting the side pressure of the nose pad as needed to provide comfortable compression on the nasal region.

In some aspects, adjusting the shape of the sponge-like insert comprises manipulating embedded metal wire casings to fit the specific contour of the nose of the user.

In some aspects, the method further includes cleaning the sponge-like insert, cooling gel, and nose pad at regular intervals using a recommended cleaning solution to maintain hygiene.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a perspective view of a post-surgical rhinoplasty compression mask, in accordance with a first illustrative embodiment of the invention;

FIG. 2 presents an exploded view of the post-surgical rhinoplasty compression mask illustrated in FIG. 1, shown with the component parts including a hard plastic shell forming the exterior of the mask;

FIG. 3 presents a side perspective view of the proximal end of the post-surgical rhinoplasty compression mask illustrated in FIG. 1, shown with particular emphasis on the side Velcro straps that go about the side of a user's face; and

FIG. 4 presents a rear view of the post-surgical rhinoplasty compression mask illustrated in FIG. 1.

Like reference numerals refer to like parts throughout the several views of the drawings.

### DETAILED DESCRIPTION

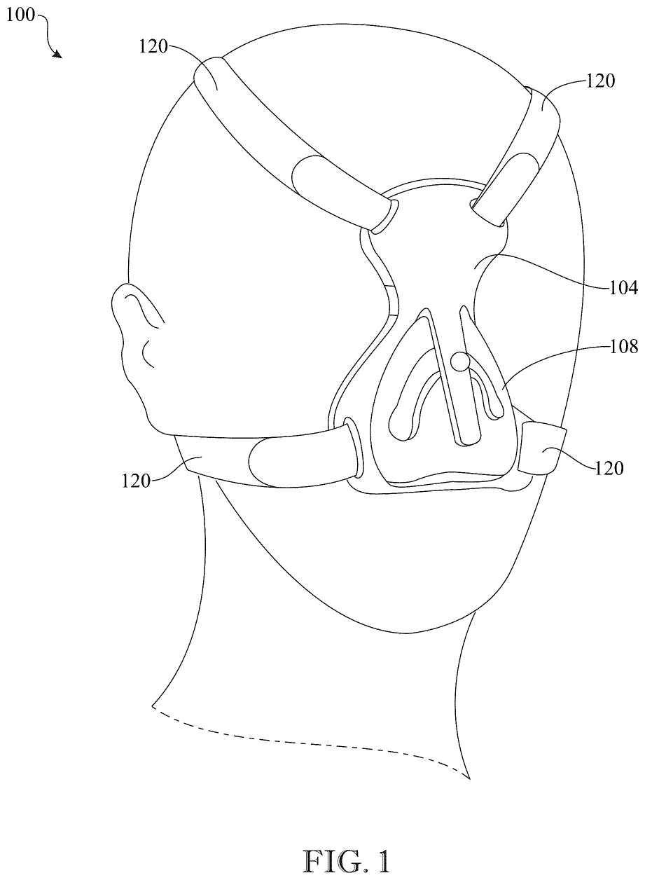

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The post-surgical rhinoplasty compression mask, delineated by numeral 100 and portrayed in FIG. 1, emerges as a groundbreaking response to the challenges faced by patients during the post-operative recovery phase following nasal surgeries. As modern medicine advances, there is an increasing need for post-operative solutions that seamlessly combine therapeutic benefits with user comfort. The mask 100 is conceptualized and designed to address this very gap.

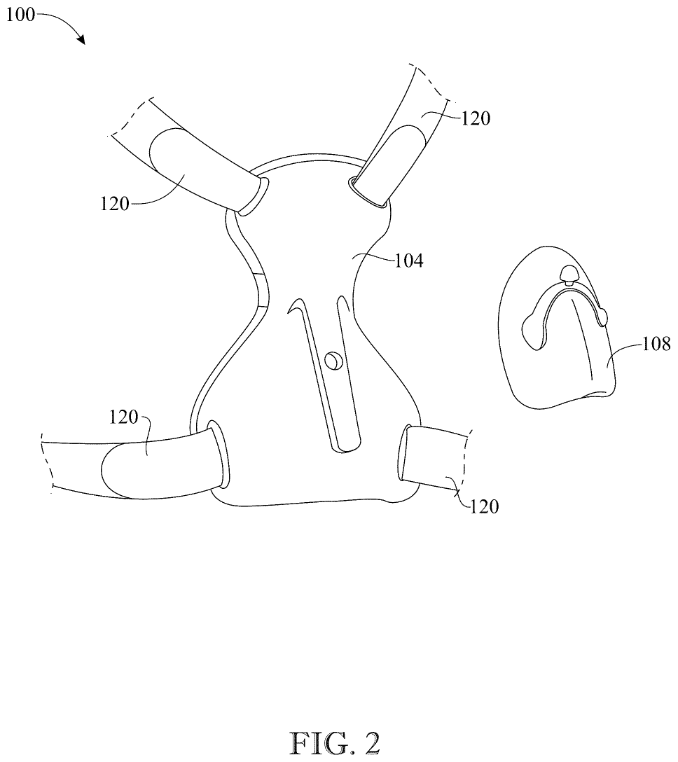

At the forefront of the mask's design is its hard plastic shell 104, vividly presented in FIG. 2. Carefully constructed to be lightweight yet durable, this shell ensures that users experience minimal discomfort, even during extended periods of wear. The selection of plastic is not just a matter of structural integrity but also user safety. By opting for hypoallergenic materials such as polypropylene or ABS (acrylonitrile butadiene styrene), the mask ensures minimal chances of skin irritations, catering to users with sensitive skin. The contour-centric design of the shell 104 ensures it adheres effortlessly to the diverse range of human facial structures, providing an unparalleled balance between form and function.

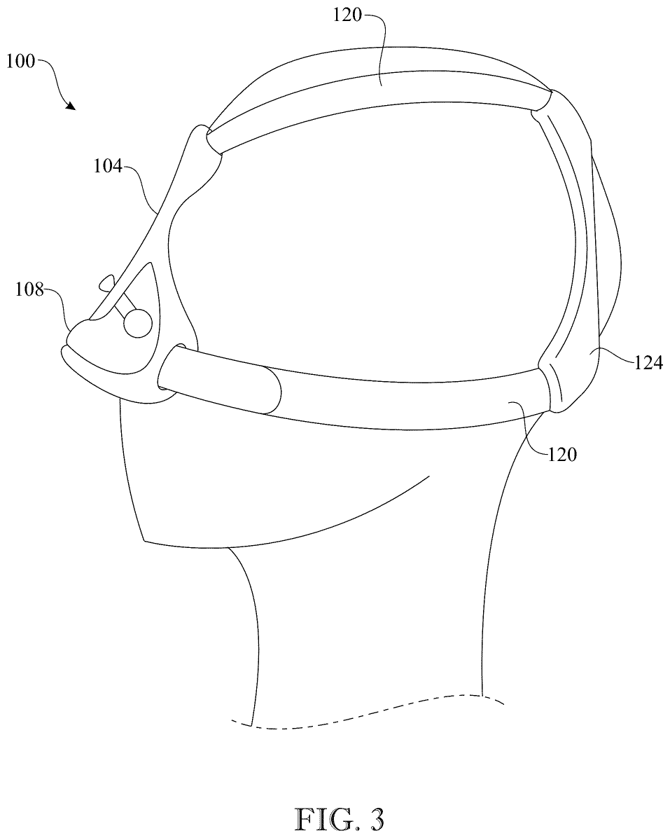

The mask 100 includes a removable sponge-like insert 108 that is placed within the plastic shell 104 (FIG. 3). This insert 108 is made from a soft, compressible, and breathable material such as foam, which facilitates a comfortable fit and adequate airflow to reduce heat buildup and perspiration. This insert 108 can be customized based on the user's face shape, helping to ensure that the mask 100 provides a consistently secure and snug fit.

The sponge-like insert 108 incorporates a series of metal wire casings (not shown in the figures). These casings provide a degree of flexibility to the insert 108, enabling the user to adjust the mask 100 to their unique facial contours. The wire casings can be made from rust-resistant metals such as stainless steel or titanium. Alternatively, they could be formed from flexible, biocompatible plastic materials.

In some embodiments, the foam insert 108 can be replaced by a cooling gel insert (not shown in the figures). Recognizing that post-operative care is dynamic and the needs of a patient change as they progress through the recovery, the design of the mask is astutely crafted to permit users to alternate between the foam insert 108 and the cooling gel insert.

In the immediate aftermath of surgery, the cooling gel insert may become paramount. Ensconced in a leak-proof pouch, this gel, when chilled, provides a soothing cold compression, effectively mitigating post-surgical swelling and discomfort. As days progress and the need for cooling diminishes, users can effortlessly transition to the foam insert 108, ensuring a continued snug fit without the coldness.

In some embodiments, the mask may comprise a removable nose pad with adjustable side pressure capabilities. The nose pad, which can be constructed from soft and skin-friendly materials such as silicone or memory foam, offers additional comfort and support to the user's nose. The adjustable side pressure can be controlled through an embedded mechanical system such as tension springs, screw mechanisms, or air-filled bladders that can be manually inflated or deflated. This allows for custom pressure adjustment to the sides and bridge of the user's nose, further enhancing the healing process.

The mask 100 is secured on the user's face with Velcro-equipped straps 120 (FIG. 3). These straps, which may be made from a strong, flexible material such as nylon, form a network around the plastic shell 104 and the sponge-like insert 108. The Velcro fasteners allow for easy adjustability, catering to a wide range of head sizes. The placement of the straps 120 can also be designed to ensure that the mask 100 allows unrestricted vision, breathing, and oral communication while being worn.

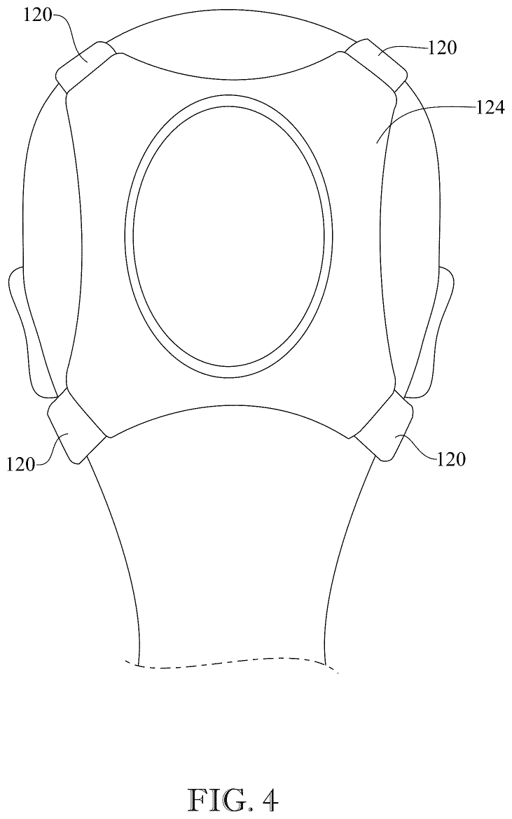

FIG. 4 is a rear visualization of mask 100. The straps 120 offer individual adjustability, converging harmoniously at the back, ensuring a cohesive, uniform, and comfortable fit. The network of these straps has been designed to optimize pressure distribution, ensuring that no single area faces undue stress.

As can be further seen in FIGS. 3 and 4, each of the straps 120 are attached to a structure 124 that covers the majority of the back of the head of the user. In some embodiments, the straps 120 may be removably attached to the structure 124, whereas in other embodiments, the straps 120 may be fixedly attached to the structure 124. In some embodiments, the structure 124 may be a meshed structure that is soft and does not apply much pressure on the head of the user and thus makes it comfortable for the user to wear the mask 100. In other embodiments, the structure 124 comprises foam so that the mask 100 when worn by the user does not apply pressure at the back of the head of the user. Moreover, the structure 124 ties all other components of the mask 100 such that the mask 100 when worn by the user fits snugly but comfortably.

The assembly of the mask 100 is designed to be straightforward and user-friendly. Each component—the plastic shell 104, the sponge-like insert 108 with its wire casings, the Velcro straps 120, the structure 124 can be individually removed and replaced. This allows for regular maintenance, cleaning, and sterilization, contributing to the overall hygiene and performance of the mask 100. The mask 100 and its individual components can be safely stored and transported in a custom-made carrying case, ensuring the durability and longevity of the mask 100. This design consideration also augments the longevity of the mask, as users can replace specific components instead of the entire mask.

Alternative embodiments of the post-surgical rhinoplasty compression mask 100 could feature a variety of modifications and additional functionalities to further enhance usability and therapeutic outcomes.

For instance, the mask 100 could incorporate an electronic component for automated control of pressure application. In this embodiment, sensors embedded in the insert 108 could relay real-time data to a microprocessor within the mask 100, which would then dynamically adjust the pressure exerted by the mask 100 based on the healing stage, patient comfort level, or specific instructions from the healthcare provider.

Another alternative embodiment could include a heating feature in addition to the cooling gel. A removable heating insert could be used to provide warm compress therapy, which is often beneficial in the later stages of the healing process. This feature could be controlled by a temperature regulation system to ensure safe and effective application of heat.

Additionally, in another embodiment, the mask 100 could feature an integrated pulse oximeter or similar sensor for real-time monitoring of blood oxygen levels in the nasal tissue. This could provide valuable feedback to healthcare providers and users about the healing progress and enable timely interventions if necessary.

Finally, the mask 100 could also be offered as part of a comprehensive post-surgery care kit, which would include additional items such as extra cooling gel packs, replacement sponge-like inserts 108, cleaning solutions, and a user manual with detailed instructions and guidance for use, maintenance, and troubleshooting. Such a kit would provide a one-stop solution for patients, further enhancing convenience and promoting adherence to post-surgical care guidelines.

In conclusion, the present invention offers an innovative and comprehensive solution for post-surgical rhinoplasty care. By seamlessly integrating various therapeutic features into a single device, the post-surgical rhinoplasty compression mask 100 significantly simplifies and enhances the recovery process. The design of the mask 100 and its components offer a user-friendly, customizable, and effective solution, marking a considerable improvement over traditional post-surgical care methods.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A post-surgical rhinoplasty compression mask, comprising:
a hard plastic shell forming an exterior structure of the mask;
a removable insert designed to fit within the plastic shell; and
a plurality of straps integrated with the plastic shell, the plurality of straps designed to surround a face and back of a head of a user that wears the mask, providing support and stability to the mask and allowing size adjustability to fit different head sizes.

2. The mask of claim 1, wherein the removable insert is a sponge-like insert comprising a series of metal wire casings, allowing the insert to be shaped to the contour of a nose of the user.

3. The mask of claim 2, wherein the sponge-like insert comprises a breathable material.

4. The mask of claim 1, wherein the removable insert is a cooling gel insert.

5. The mask of claim 4, wherein the cooling gel insert comprises a removable cooling gel enclosed in a leak-proof container.

6. The mask of claim 1, wherein the plurality of straps comprise Velcro fasteners.

7. The mask of claim 1, further comprising a user-adjustable chin strap for additional support and stability.

8. The mask of claim 7, wherein the straps are adjustable in length.

9. The mask of claim 1, further comprising a mesh structure to support back of a head of the user.

10. The mask of claim 1, further comprising a foam structure to support back of a head of the user.

11. A method of using a post-surgical rhinoplasty compression mask, the method comprises:
inserting a removable sponge-like insert in a hard plastic shell of the mask; and
securing the mask onto a face of a user using straps equipped with Velcro fasteners, wherein the straps surround the face and back of a head of the user, providing support and stability while allowing for adjustability to fit different head sizes.

12. The method of claim 11, further comprising:
replacing the sponge-like insert with a cooling gel insert that comprises cooling gel to provide a cooling effect.

13. The method of claim 12, further comprising:
periodically checking the cooling gel to maintain consistent temperature and replacing the cooling gel, as necessary.

14. A kit, comprising:
a post-surgical rhinoplasty compression mask, comprising:
a hard plastic shell forming an exterior structure of the mask;
a removable insert designed to fit within the plastic shell; and
a plurality of straps integrated with the plastic shell, the plurality of straps designed to surround a face and a back of head of a user that wears the mask, providing support and stability to the mask and allowing size adjustability to fit different head sizes;
a manual to instruct a user on how to use and adjust the mask; and
a carrying case for storage and transportation of the mask.

15. The kit of claim 14, wherein the removable insert comprises a sponge-like insert or a cooling gel insert.

16. The kit of claim 15, further comprising: one or more replacement sponge-like inserts, one or more replacement cooling gel inserts, and a cleaning solution for the mask.

17. The kit of claim 14, wherein the plurality of straps comprise Velcro fasteners.

18. The kit of claim 14, wherein the mask comprises a user-adjustable chin strap for additional support and stability.

19. The kit of claim 14, wherein the straps are adjustable in length.

20. The kit of claim 14, wherein the plurality of straps extend to form a structure supported by back of a head of the user.
